(19) United States
(12) Patent Application Publication
(10) Pub. No.: US 2010/0130909 A1
(43) Pub. Date: May 27, 2010

(54) TREATMENT OF BARRETT'S ESOPHAGUS USING PHOTODYNAMIC THERAPY

(75) Inventors: Ravindra K. Pandey, Williamsville, NY (US); Thomas J. Dougherty, Grand Island, NY (US); David A. Bellnier, Buffalo, NY (US); Hector R. Nava, Williamsville, NY (US)

Correspondence Address: MICHAEL L. DUNN, SIMPSON & SIMPSON, PLLC, 5555 MAIN STREET, WILLIAMSVILLE, NY, 14221, US

(73) Assignee: HEALTH RESEARCH, INC., Buffalo, NY (US)

(21) Appl. No.: 12/448,662

(22) PCT Filed: Sep. 27, 2007
(86) PCT No.: PCT/US2007/020817

§ 371 Date: Feb. 4, 2010

Related U.S. Application Data
(60) Provisional application No. 60/879,474, filed on Jan. 9, 2007.

(51) Int. Cl. A61M 37/00; A61K 31/409

(52) U.S. Cl. 604/20; 514/410

(57) ABSTRACT

A method for treatment of Barrett's esophagus comprising the steps of: injecting HPPH in a physiologically compatible medium into a patient having Barrett's esophagus tissue to provide a dose level of 3 through 5 mg/m² of body surface area, waiting for a time period of 24 through 60 hours to permit preferential absorption of the HPPH into Barrett's esophagus tissue, and exposing the Barrett's esophagus tissue to light at a wavelength of about 670±5 nm at an energy of from about 75 to about 200 Joules/cm.

### CROSS REFERENCE TO RELATED PATENT APPLICATION

This application claims priority from U.S. Provisional Application No 60/879,474, filed 9 Jan. 2007.

### STATEMENT REGARDING FEDERALLY SPONSORED RESEARCH

This invention was made with funding from the National Institute of Health Grant Numbers NIH (1R21 CA109914-01 and CA 55792). The United States Government may have certain rights in this invention.

### BACKGROUND OF THE INVENTION

Barrett's esophagus is characterized by development of abnormal tissue, usually as a protective response to erosion of the esophagus by acid reflux. While Barrett's esophagus is not a well known disease, associated acid reflux has become well known by advertisement of pills for its treatment. Unfortunately, while such pills can in fact help prevent acid reflux, they do little or nothing to treat already developed Barrett's esophagus. While Barrett's esophagus itself may or may not be uncomfortable or painful, its presence is a risk factor for development of cancer of the esophagus that is debilitating and can be life threatening. Removal of Barrett's esophagus has not been readily accomplished. Experimental approaches include endoscopic ablation by RF energy or heat, and endoscopic mucosal removal, all usually limited to small segments of Barrett's esophagus.

For the last several years porphyrin-based compounds have been used for the treatment of cancer by photodynamic therapy (PDT). The concentration of certain porphyrins and related tetrapyrrolic systems is higher in malignant tumors than in most normal tissues and that has been one of the main reason for using these molecules as photosensitizers. Some tetrapyrrole-based compounds have been effective in a wide variety of malignancies, including skin, lung, bladder, head and neck and esophagus. There have, however been associated problems with their use including skin phototoxicity, normal tissue damage, insufficient depth of penetration and a high percentage of esophageal strictures. The precise mechanism(s) of PDT are unknown; however, in vivo animal data suggests that both direct cell killing and loss of tumor vascular function play a significant role. A new and well tested tetrapyrrolic compound is the 3-(1-hexyloxy) ethyl-derivative of pyropheophorbide-a (HPPH). HPPH, as used herein, means the 3-(1-hexyloxy) ethyl-derivative of pyropheophorbide-a in both its free acid and ester and salt forms. This compound is tumor-avid and has undergone Phase I/II human clinical trials at the Roswell Park Cancer Institute in Buffalo, New York.

Photodynamic therapy (PDT) is believed to exploit the biological consequences of localized oxidative damage inflicted by photodynamic processes. Three critical elements required for initial photodynamic processes to occur are: a photosensitizer, light at the photosensitizer-specific absorption frequency or wavelength, and oxygen. The light at the required wavelength is believed to trigger singlet oxygen production to destroy tissue in which it is concentrated.

Tetrapyrollic photosensitizers, such as the photosensitizer porfimer sodium, sold under the trademark PHOTOFRIN™, and HPPH, concentrate well in most tumor tissue.

Barrett's esophagus is associated with an increased occurrence of mucosal dysplasia and esophageal cancer (Overholt et al., Gastrointestinal Endoscopy, volume 49:1-7, 1999; volume 62:488-498, 2005; unpublished observations by present inventors). Photodynamic therapy using Porfimer sodium has been found to be a nonsurgical therapy that eliminates or reduces the extent of Barrett's mucosa, thus reducing the risk of development of esophageal cancer.

Unfortunately, the use of porfimer sodium to treat Barrett's esophagus has a number of serious side effects including long term sensitivity to light, especially sunlight, and injury to surrounding normal tissue, especially the formation of esophageal strictures.

A review of published literature (Overholt et al., Gastrointestinal Endoscopy, volume 49:1-7, 1999; volume 62:488-498, 2005) and non-published sources, not necessarily prior art to the present invention, indicate that the use of porfimer sodium at its optimized dose level of 2 mg/kg and activation at its preferential light absorption wavelength of 630 nm, and light exposure of 100 to 250 J/cm resulted in replacement of 75-80% of Barrett's mucosa with normal esophageal mucosa in all patients treated (100 patients). Complete ablation of Barrett's mucosa was observed in 43% of patients. Of these, 8% percent achieved complete ablation of Barrett's mucosa with PDT treatment only, while 35% required thermal ablation to destroy small residual islands of abnormal mucosa. Unfortunately, esophageal strictures occurred in 34% of all patients treated.

The use of HPPH for treatment of obstructive esophageal cancer has been described. (Optical Methods for tumor Treatment and Detection: Mechanisms and Techniques in Photodynamic Therapy IX, Thomas Dougherty, Editor, Proceedings of SPIE Vol. 3909 (2000). This document does not describe effects upon Barrett's esophagus.

### BRIEF DESCRIPTION OF THE INVENTION

In accordance with the invention, we have discovered that HPPH, like porfimer sodium, also ablates Barrett's esophagus when combined with exposure of such tissue to light at the preferential absorption wavelength of HPPH (670±5 nm). However, it has been surprisingly discovered that HPPH accomplishes the desired result with higher success at lower dosages and importantly with fewer esophageal strictures. HPPH is effective at doses of only 0.08 to 0.13 mg/kg of body weight (3-5 mg/m² of body surface) versus a minimum of 2 mg/kg of body weight for porfimer sodium.

HPPH, i.e. 3-(1-hexyloxy) ethyl-derivative of pyropheophorbide-a, has the following formula:

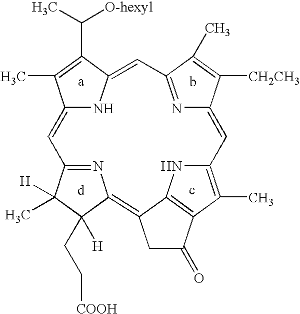

and includes the salts and alkyl esters thereof and may be prepared as set forth in U.S. Pat. Nos. 5,198,460 and 5,314,905 reissued as RE39094 and RE38994 respectively, all of which are incorporated herein by reference.

The method of the invention includes the steps of:

injecting HPPH in a physiologically compatible medium into a patient having Barrett's esophagus tissue to provide a dose level of 3 through 5 mg/m² of body surface area, preferably 3 through 4 mg/m² of body surface area,

waiting for a time period of 24 through 60 hours to permit preferential absorption of the

HPPH into Barrett's esophagus tissue, and exposing the Barrett's esophagus tissue to light at a wavelength of about 670±5 nm at an energy of from 75 to 200 Joules/cm, preferably 75 to 200 Joules/cm.

### DETAILED DESCRIPTION OF THE INVENTION

Injection of the HPPH is preferably accomplished intravenously usually over a time period of 0.75 to 3 hours in a physiologically compatible medium. The time period is functionally dependent upon rate of infusion and dose level desired. The concentration is preferably 0.5 through 1.5 mg/ml in medium and the medium is preferably 0.1% polysorbate 80, 2% ethyl alcohol and 5% glucose in normal saline.

Exposure is accomplished using a fiber optic carrying laser light emitted by a laser. The laser may be any suitable laser emitting light at the wavelength and energy desired, e.g. a dye or diode laser. Exposure may be adjusted by length of time of exposure and/or adjustment of light intensity.

Using the above parameters, a phase I/II trial using HPPH and a phase III trial using PHOTOFRIN™, the latter approved by the United States Food and Drug Administration, the following results for response of Barrett's esophagus were obtained. “CR” as used in this context means that no Barrett's esophagus tissue remained after treatment and does not indicate that there was no later relapse.

| | |
|---|---|
| PHOTOFRIN | HPPH |
| 100 patients | 30 patients |

Treatment Response

| | | | |
|---|---|---|---|
| PDT ONLY | 8% CR | PDT ONLY | 4% CR |
| (compilation of 1-3 | | (only 1 PDT | |
| PDT treatments) | | treatment) | |
| PDT + NdYAG | 35% CR | PDT ONLY | 47% (>90 to <100% |
| (compilation of 1-3 | | (only 1 PDT | ablation) |
| PDT treatments + | | treatment; does | |
| thermal ablation) | | not include | |
| | | NdYAG thermal | |
| | | ablation) | |

Complications

| | | | |
|---|---|---|---|
| ESOPHAGEAL | 34% | ESOPHAGEAL | 12% |
| STRICTURE | | STRICTURE | |
| (compilation of 1-3 | | (only 1 PDT | |
| PDT treatments) | | treatment) | |
| SUNBURN | 25-30% | SUNBURN | <7% |

In view of the above, it is clear that HPPH is as effective or more effective than PHOTOFRIN™ in treating Barrett's esophagus at lower dose even when a single treatment with HPPH is used as compared with responses with a combination of single and repeated treatments with PHOTOFRIN™ apparently even without subsequent thermal ablation with HPPH. Even better results using HPPH can be expected with repeated treatment and/or thermal ablation.

What is claimed is:

1. A method for treatment of Barrett's esophagus comprising the steps of:
injecting HPPH in a physiologically compatible medium into a patient having Barrett's esophagus tissue to provide a dose level of 3 through 5 mg/m² of body surface area,
waiting for a time period of 24 through 60 hours to permit preferential absorption of the HPPH into Barrett's esophagus tissue, and
exposing the Barrett's esophagus tissue to light at a wavelength of about 670±5 nm at an energy of from about 75 to about 200 Joules.

2. The method of claim 1 where the dose level of HPPH is 3.0 to 4.0 mg/m².

3. The method of claim 1 where the energy is from about 75 to about 150 Joules.

4. The method of claim 1 where the waiting time is from about 24 to about 60 hours.
